SEQUENCE LISTING (2 sequences)

SEQ ID NO: 1 - DNA, 1098 bp, Rhodococcus rhodochrous
atggtcgaatacacaaacacattcaaagttgctgcggtgcaggcacagcctgtgtggttcgacgcggccaaaacggtcgacaagaccgtgtccatcatcgcggaagcagcccggaacgggtgcgagctcgttgcgtttcccgaggtattcatcccggggtacccgtaccacatctgggtcgacagcccgctcgccggaatggcgaagttcgccgtgcgctaccacgagaattccctgacgatggacagcccgcacgtacagcggttgctcgatgccgcccgcgaccacaacatcgccgtagtggtgggaatcagcgagcgggatggcggcagcttgtacatgacccagctcatcatcgacgccgatgggcaactggtcgcccgacgccgcaagctcaagcccacccacgtcgagcgttcggtatacggagaaggaaacggctcggatatctccgtgtacgacatgcctttcgcacggcttggcgcgctcaactgctgggagcatttccagacgctcaccaagtacgcaatgtactcgatgcacgagcaggtgcacgtcgcgagctggcctggcatgtcgctgtaccagccggaggtccccgcattcggtgtcgatgcccagctcacggccacgcgtatgtacgcactcgagggacaaaccttcgtggtctgcaccacccaggtggtcacaccggaggcccacgagttcttctgcgagaacgaggaacagcgaaagttgatcggccgaggcggaggtttcgcgcgcatcatcgggcccgacggccgcgatctcgcaactcctctcgccgaagatgaggaggggatcctctacgccgacatcgatctgtctgcgatcaccttggcgaagcaggccgctgaccccgtgggccactactcacggccggatgtgctgtcgctgaacttcaaccagcgccgcaccacgcccgtcaacaccccactttccaccatccatgccacgcacacgttcgtgccgcagttcggggcactcgacggcgtccgtgagctcaacggagcggacgaacagcgcgcattgccctccacacattccgacgagacggaccgggcgacagcctccatc

SEQ ID NO: 2 - protein, 366 aa, Rhodococcus rhodochrous
MVEYTNTFKVAAVQAQPVWFDAAKTVDKTVSIIAEAARNGCELVAFPEVFIPGYPYHIWVDSPLAGMAKFAVRYHENSLTMDSPHVQRLLDAARDHNIAVVVGISERDGGSLYMTQLIIDADGQLVARRRKLKPTHVERSVYGEGNGSDISVYDMPFARLGALNCWEHFQTLTKYAMYSMHEQVHVASWPGMSLYQPEVPAFGVDAQLTATRMYALEGQTFVVCTTQVVTPEAHEFFCENEEQRKLIGRGGGFARIIGPDGRDLATPLAEDEEGILYADIDLSAITLAKQAADPVGHYSRPDVLSLNFNQRRTTPVNTPLSTIHATHTFVPQFGALDGVRELNGADEQRALPSTHSDETDRATASI

(19) United States
(12) Patent Application Publication
(10) Pub. No.: US 2009/0130726 A1
(43) Pub. Date: May 21, 2009

(54) PROCESS FOR CONVERTING AROMATIC HALO-SUBSTITUTED DINITRILES INTO HALO-SUBSTITUTED CYANOCARBOXYLIC ACIDS

(76) Inventors: Andrew Wells, Leicestershire (GB); Stefaan De Wildeman, Heerlen (NL)

Correspondence Address: BIRCH, STEWART, KOLASCH & BIRCH, LLP, P.O. BOX 747, FALLS CHURCH, VA, 22040-0747, US

(21) Appl. No.: 12/066,368

(22) PCT Filed: Sep. 20, 2006
(86) PCT No.: PCT/SE2006/001074

§ 371 Date: Mar. 10, 2008

(30) Foreign Application Priority Data
Sep. 22, 2005 (SE) 0502100-1

(51) Int. Cl. C12P 13/00

(52) U.S. Cl. 435/128

(57) ABSTRACT

The present invention is directed to a process for converting aromatic halo-substituted dinitriles into the corresponding cyanocarboxylic acids in the presence of a nitrilase.

### FIELD OF THE INVENTION

The present invention is directed to a process for converting aromatic halo-substituted dinitriles into the corresponding cyanocarboxylic acids in the presence of a nitrilase.

### BACKGROUND OF THE INVENTION

Hydrolysis of a cyano group of a nitrile compound to obtain the corresponding carboxylic acids is a common method for obtaining carboxylic acids. However, selective hydrolysis reactions of a cyano group in a chemical synthesis are generally complicated procedures. Protection of a specific cyano group is often necessary. Instead, bioreactions in which only a part of the cyano groups of a polynitrile compound are hydrolysed to obtain the corresponding cyano carboxylic acid are commonly used.

Bioreactions are generally admitted to have high selectivity. However, they are in many cases accompanied by production of impurities due to side reactions, often caused by the use of wild-type microorganisms.

Nitrilase is an enzyme having nitrilase activity, i.e. an enzyme that catalyzes a reaction where a nitrile compound is converted into a carboxylic acid. Microorganisms that produce this enzyme include Fusarium solani (see Biochem. J. 167, 685-692 (1977)), Nocardia sp. (see, Int. J. Biochem., 17, 677-683 (1985)), Arthrobacter sp. (see, Appl. Environ, Microbiol., 51, 302-306 (1986)), Rhodococcus rhodochrous J1 (see, Eur. J. Biochem., 182, 349-356 (1989)), Rhodococcus rhodochrous K-22 (see, J. Bacteriol, 172, 4807-4815 (1990)), Rhodococcus rhodochrous PA-34 (see, Appl. Microbiol. Biotechnol., 37, 184-190 (1992)) and Rhodococcus sp. ATCC39484 (see, Biolechnol. Appl. Biochem., 15, 283-302 sp. (1992)).

In recent years, attempts have been made to utilize these microorganisms' capability of converting nitrile compounds. Among these microorganisms, the Rhodococcus sp. ATCC39484 strain has been reported to have a capacity to hydrolyze aromatic polynitrile compounds having a plurality of nitrile groups selectively. However, the nitrilase of this microorganism has been reported to be relatively low in the activity on aromatic polynitrile compounds (EP1142997 A1).

U.S. Pat. No. 4,629,700 discloses a process for producing aromatic acids by biological hydrolysis of the corresponding nitrites. EP 178106 discloses a process for producing cyano carboxylic acids by the selective hydrolysis of a cyano group from a dinitrile compound using a mononitrilase of bacterial origin.

EP1142997 A1 discloses a novel Rhodococcus bacterium and a process for hydrolyzing a cyano group of a nitrile compound to produce the corresponding carboxylic acid.

EP444640 A2 discloses a process for conversion of a nitrile into the corresponding carboxylic acid using a nitrilase of a Rhodococcus strain cultured in the presence of a lactam compound.

Appl. Microbiol. Biotechnol. 29 (1988), 231-233 discloses the of Rhodococcus rhodochrous nitrilase J1 to catalyze the reaction converting 1,3-dicyanobenzene into 3-cyanobenzoic acid.

Eur. J. Biochem. 182 (1989), 349-356 discloses the use of Rhodococcus rhodochrous J1 nitrilase to catalyze the conversion of mononitriles into the corresponding carboxylic acids. According to the authors, the enzyme exhibited little detectable activity when aromatic halo-substituted nitrites were used as substrate.

The object of the present invention is to improve the enzyme-catalyzed reaction of converting halo-substituted aromatic dinitrile compounds into the corresponding cyanobenzoic acids by increasing the activity and selectivity, thereby making the reaction suitable for industrial scale.

### SUMMARY OF THE INVENTION

The present invention provides a process for converting nitriles of formula I into carboxylic acids of formula II,

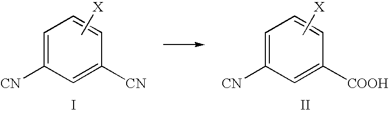

wherein X is halogen;
said conversion being performed in the presence of a nitrilase.

In one embodiment of the present invention, the halogen X is in the 5-position of the compounds of formula I and formula II respectively. In a further embodiment of the present invention, X is fluoro. In yet a further embodiment of the present invention, 5-fluoro-1,3-dicyanobenzene is converted into 5-fluoro-3-cyano-benzoic acid.

In one embodiment of the present invention, the nitrilase is a wild-type nitrilase. In another embodiment of the present invention, the nitrilase is a cloned nitrilase. In a further embodiment, the nitrilase is a Rhodococcus nitrilase. In yet a further embodiment, the nitrilase is a Rhodococcus rhodochrous nitrilase. In another embodiment, the nitrilase is a nitrilase encoded by the nucleic acid sequence SEQ ID No. 1. In yet another embodiment, the nitrilase has the amino acid sequence SEQ ID No. 2.

The invention also relates to nucleic acid sequences that code for a polypeptide having nitrilase activity, to nucleic acid constructs comprising the nucleic acid sequences, and to vectors comprising the nucleic acid sequences or the nucleic acid constructs. The invention further relates to amino acid sequences that are encoded by the nucleic acid sequences, and to microorganisms comprising the nucleic acid sequences, the nucleic acid constructs or vectors comprising the nucleic acid sequences or the nucleic acid constructs.

### DETAILED DESCRIPTION OF THE INVENTION

The process according to the present invention may be carried out at a pH of from 4 to 11, in particular from 4 to 9. In one embodiment, the process is performed at a pH of about 7.

It is possible to use from 0.01 to 25% by weight of nitrile in the process. In one embodiment, 0.1 to 10% by weight of nitrile is used. In a further embodiment, 0.5 to 5% by weight of nitrile is used. Different amounts of nitrile can be used in the reaction depending on the nitrite. The smallest amounts (equals amounts between 0.01 to 5% by weight) of nitrile may be used in the case of nitrites (cyanohydrins) that are in equilibrium with the corresponding aldehydes and hydrocyanic acid.

Usually, the substrate concentration, i.e. the concentration of the compound of formula I in the reaction mixture is within the range of from 20 g/l to 40 g/l.

In the context of the present invention, halogen is fluoro, chloro, bromo or iodo.

In the context of the present invention, selectivity is defined as the proportion of the total amount of product that is the desired product.

In the context of the present invention, regioselectivity is the selective hydrolysis of only one nitrile group of the compound of formula I into the corresponding carboxylic acid of formula II.

The process according to the invention may be carried out at a temperature between 0° C. to 80° C. In one embodiment, the reaction is performed between 10° C. to 60° C. In a further embodiment, the reaction is carried out between 15° C. to 50° C.

In the process it is possible to obtain an overall activity of at least from 0.1 up to 5 U/mgₙᵢₜᵣᵢₗₐₛₑ. In one embodiment, the overall activity is from 0.1 up to 0.2 U/mgₙᵢₜᵣᵢₗₐₛₑ. In one embodiment, the overall activity is from 0.5 up to 2 U/mgₙᵢₜᵣᵢₗₐₛₑ. The overall activity is determined after 95% conversion of the substrate.

It is possible to use growing cells which comprise the nucleic acids, nucleic acid constructs or vectors according to the invention for the process according to the invention. Resting or disrupted cells can also be used. Disrupted cells mean, for example, cells that have been made permeable by a treatment with, for example, solvents, or cells that have been disintegrated by an enzyme treatment, by a mechanical treatment (e.g. French press or ultrasound) or by any other method. The crude extracts obtained in this way may be used in the process according to the invention. Purified or partially purified enzymes can also be used for the process. Immobilized microorganisms or enzymes can likewise be used in the reaction.

The carboxylic acids prepared in the process according to the invention can be isolated from the aqueous reaction solution by extraction or crystallization or by extraction and crystallization. For this purpose, the aqueous reaction solution is acidified with an acid such as a mineral acid (e.g. HCl or H₂SO₄) or an organic acid, advantageously to pH values below 2, and then extracted with an organic solvent. The extraction can be repeated several times to increase the yield. Organic solvents that can be used are in principle all solvents that show a phase boundary with water, where appropriate after addition of salts. Possible solvents are solvents such as toluene, benzene, hexane, methyl tert-butyl ether or ethyl acetate. The products can also be purified by binding to an ion exchanger and subsequently eluting with a mineral acid or carboxylic acid such as HCl, H₂SO₄, formic acid or acetic acid. Alternatively, the product may be crystallized and isolated directly from the reaction mixture according to standard procedures.

After concentration of the aqueous or organic phase, the products can usually be isolated in good chemical purities, meaning a chemical purity of greater than 90%. After extraction, the organic phase with the product can, however, also be only partly concentrated, and the product can be crystallized. For this purpose, the solution may be cooled to a temperature of from 0° C. to 10° C. The crystallization can also take place directly from the organic solution. The crystallized product can be taken up again in the same or a different solvent for renewed crystallization and be crystallized once again.

The carboxylic acids can, however, also be crystallized out of the aqueous reaction solution immediately after acidification with an acid to a pH below, for instance, 2. This may entail the aqueous solution being concentrated by heating to reduce its volume by 10 to 90%. In one embodiment, the volume is reduced by 20 to 80%. In a further embodiment, the volume is reduced to 30 to 70%. The crystallization may be carried out with cooling, for instance at temperatures between 0° C. and 10° C.

With these types of workup, the product of the process according to the invention can be isolated in yields of from 60% to 100%. In one embodiment, the yield is from 80% to 100%. In a further embodiment, the yield is from 90% to 100%, based on the nitrile employed for the reaction. In yet another embodiment the yield is from 95% to 100%. In a further embodiment, the yield is from 98% to 100%. In one embodiment, the isolated product has a chemical purity of >90%. In a further embodiment, the purity is >95%. In yet another embodiment the purity is >98%.

The selectivity of the process according to the present invention is usually within the range of from 90% to 100%.

The products obtained in this way may be used as starting material for organic syntheses to prepare drugs or agrochemicals.

A person skilled in the art can produce the nucleic acid sequence SEQ. ID No. 1, which encodes a Rhodococcus nitrilase, by synthesising and expressing the nitrilase by methods known in the art (e.g. Sambrook et al. “Molecular Cloning”, Cold Spring Harbor Laboratory, 1989). SEQ ID No. 2 illustrates the corresponding amino acid sequence.

The nucleic acid construct according to the invention means the nitrilase gene of sequence according to SEQ ID No. 1, which has been functionally linked to one or more regulatory signals to increase gene expression. These regulatory sequences are, for example, sequences to which the inducers or repressors bind and thus regulate the expression of the nucleic acid. In addition to these novel regulatory sequences, it is also possible for the natural regulation of these sequences to be present in front of the actual structural genes and, where appropriate, to have been genetically modified so that the natural regulation is switched off and the expression of the genes has been increased. The nucleic acid construct may, however, also have a simpler structure, that is to say no additional regulatory signals have been inserted in front of the sequence SEQ ID No. 1, and the natural promoter with its regulation has not been deleted. Instead, the natural regulatory sequence is mutated in such a way that the regulation no longer takes place, and gene expression is increased. The nucleic acid construct may additionally comprise one or more enhancer sequences, functionally linked to the promoter, which make increased expression of the nucleic acid sequence possible. The nucleic acids according to the invention may be present in one or more copies in the construct. The construct may also comprise further markers such as antibiotic resistances or auxotrophy-complementing genes where appropriate for selection of the construct.

Examples of regulatory sequences for the process according to the invention are present in promoters such as cos, tac, trp, tet, trp-tet, lpp, lac, lpp-lac, lacl^{q}, T7, T5, T3, gal, trc, ara, SP6, λ-P_{R} or the λ-P_{L} promoter, which may be used in Gram-negative bacteria. Further regulatory sequences are in, for example, the Gram-positive promoters amy and SPO2, in the fungal or yeast promoters ADC1, MFα, AC, P-60, CYC1, GAPDH, TEF, rp28, ADH. Other examples in this connection are the promoters of pyruvate decarboxylase and of methanol oxidase from, for example, Hansenula. It is also possible to use artificial promoters for the regulation.

The nucleic acid construct may be inserted into a vector such as, for example, a plasmid, a phage or other DNA for expression in a host organism, which makes optimal expression of the genes in the host possible. These vectors represent a further development of the invention. Examples of such plasmids in E. coli are pLG338, pACYC184, pBR322, pUC18, pUC19, pKC30, pRep4, pHS1, pHS2, pPLc236, pMBL24, pLG200, pUR290, pIN-III¹¹³-B1, λgt11 or pBdCI, in Streptomyces are pIJ101, pIJ364, pIJ702 or pIJ361, in Bacillus are pUB110, pC194 or pBD214, in Corynebacterium are pSA77 or pAJ667, in fungi are pALS1, pIL2 or pBB116, in yeasts are 2 μM, pAG-1, YEp6, YEp13 or pEMBLYe23 or in plants are pLGV23, pGHlac⁺, pBIN19, pAK2004 or pDH51. Said plasmids represent a small selection of the possible plasmids. Further plasmids are well known to the skilled worker and can be found, for example, in the book Cloning Vectors (eds. Pouwels P. H. et al. Elsevier, Amsterdam-New York-Oxford, 1985, ISBN 0 444 904018).

The nucleic acid construct may also contain, for expression of the other genes present, in addition 3′ and/or 5′ terminal regulatory sequences to increase expression, which are selected for optimal expression depending on the selected host organism and gene or genes.

These regulatory sequences are intended to make specific expression of the genes and protein expression possible. This may mean, for example, depending on the host organism, that the gene is expressed or overexpressed only after induction, or that it is immediately expressed and/or overexpressed.

The regulatory sequences or factors may moreover influence positively, and thus increase, expression of the introduced genes. Thus, enhancement of the regulatory elements can take place at the level of transcription, by using strong transcription signals such as promoters and/or enhancers. However, it is also possible in addition to enhance translation by, for example, improving the stability of the miRNA.

In another embodiment of the vector, the vector comprising the nucleic acid construct according to the invention or the nucleic acid according to the invention can also be introduced in the form of a linear DNA into the microorganisms and be integrated by heterologous or homologous recombination into the genome of the host organism. This linear DNA may consist of a linearized vector such as a plasmid or only of the nucleic acid construct or of the nucleic acid.

For optimal expression of heterologous genes in organisms, the nucleic acid sequences may be modified to accord with the codon usage specifically used in the organism. The codon usage can easily be established on the basis of computer analyses of other known genes in the relevant organism.

Suitable host organisms for the nucleic acid according to the invention or the nucleic acid construct are in principle all procaryotic or eucaryotic organisms. The host organisms used may be microorganisms such as bacteria, fungi or yeasts. Gram-positive or Gram-negative bacteria may be used, such as bacteria of the family Enterobacteriaceae, Pseudomonadaceae, Streptomycetaceae, Mycobacteriaceae, or Nocardiaccac, particularly bacteria of the genera Escherichia, Pseudomonas, Nocardia, Mycobacterium, Streptomyces or Rhodococcus, specifically the genus and species Escherichia coli, Rhodococcus rhodochrous, Nocardia rhodochrous, Mycobacterium rhodochrous or Streptomyces lividans.

The host organism according to the invention may moreover comprise at least one proteinaceous agent for folding the polypeptides it has synthesized and, in particular, the nucleic acid sequences having nitrilase activity described in this invention and/or the genes encoding this agent, the amount of this agent present being greater than that corresponding to the basic amount in the microorganism considered. The genes coding for this agent are present in the chromosome or in extrachromosomal elements such as, for example, plasmids.

### EXAMPLES

### 1. Preparation of Cell Free Extract (CFE) Biocatalyst

A glycerol stock of E. coli DH 10B (pMS470-3-14-1-4 from a Rhodococcus rhodochrous nitrilase) was used to inoculate 1 litre Terrific Broth (TB) medium supplemented with carbenicillin (100 mg/l). After 68 hours of growth, this culture was used to inoculate 9 litre TB medium supplemented with carbenicillin (100 mg/l) and IPTG was used as inducer. After 41 hours of growth in a lab-scale fermentor, the cells were harvested by centrifugation (12227×g, 10 minutes) of full grown fermentation broth (OD₆₀₀=16.7), resuspending the cell wet weight biomass in 20 mM HEPES/NaOH buffer (pH 7.0, 13 μg benzonase per gram wet weight cells) at ratio 1 kg cells+3 kg buffer solution, and destruction of the cells in nanojet at 1600 bars (double run). 1.04 litre of CFE was obtained, with a yield of 86% (328 gram wet biomass+880 ml buffer).

### 2. Enzymatic Conversion

In a reactor 1.5 mol 5-fluoro-1,3-dicyanobenzene was suspended in 8 l 0.1 M sodium phosphate buffer holding pH=7.2. 440 ml 2.6 wt % CFE nitrilase enzyme was added to this suspension. After 72 hrs a conversion of >98% was reached at 25° C. and the reaction was stopped by addition of phosphoric acid till pH 2.4. The product crystallized and was collected by filtration. The filter cake was washed (stirred) three times with half its volume of water. Finally the product was dried in the air. Yield: 97%/1.45 mol.

### 3. Analytical Method

The conversion of 5-fluoro-1,3-dicyanobenzene to 3-cyano-5-fluoro-benzoic acid via nitrilase reaction was determined via reversed phase LC on an Inertsil ODS-3 column (50×4.6 mm I.D., 3 μm) from Varian. The compounds were eluted using a gradient of 50 mM phosphoric acid in water pH 2.7 and acetonitrile (1.0 ml/min, at 40° C.). The gradient starting conditions were 97.5/2.5 v/v % buffer/acetonitrile at time zero and the percentage acetonitrile increased to 50% in 10 min. At 10.1 min. the gradient profile returned to starting conditions. Total analysis time was 12 min. The injection volume was 5 μl and detection was performed using a spectrophotometer at UV 220 mm. Retention times for 3-cyano-5-fluoro-benzoic acid, 1,3-dicyano-5-fluoro benzene and 3-cyano-5-fluoro-benzoic acid amide were 6.75, 7.75 and 5.2 min respectively.

1. A process for conversion of a nitrile of formula I into a carboxylic acid of formula II; 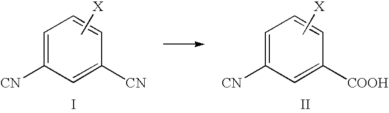
wherein X is halogen;
said conversion being performed in the presence of a nitrilase.

2. A process according to claim 1, wherein the nitrilase is a Rhodococcus nitrilase.

3. A process according to claim 2, wherein the nitrilase is a Rhodococcus rhodochrous nitrilase.

4. A process according to claim 3, wherein the nitrilase is a cloned Rhodococcus rhodochrous nitrilase.

5. A process according to claim 3, wherein the nitrilase is a wild-type Rhodococcus rhodochrous nitrilase.

6. A process according to claim 1, wherein the nitrilase is encoded by a nucleic acid sequence as depicted in SEQ ID No. 1.

7. A process according to claim 1, wherein the nitrilase has the amino acid sequence depicted in SEQ ID No. 2.

8. A process according to claim 1, wherein X is in the 5-position of the compounds of formula I and formula II.

9. A process according to claim 1, wherein X is fluoro.

10. A process according to claim 1, wherein 5-fluoro-1,3-dicyanobenzene is converted into 5-fluoro-3-cyanobenzoic acid.

11. A process according to claim 1, wherein the nitrilase is expressed in a bacterium selected from any one of the genera Escherichia, Rhodococcus, Nocardia, Streptomyces and Mycobacterium.

12. A process according to claim 11, wherein the nitrilase is expressed in Escherichia coli.

13. A process according to claim 1, wherein the process is carried out in an aqueous reaction solution.

14. A process according to claim 1, wherein the reaction is carried out at a pH of from 4 to 11.

15. A process according to claim 1, wherein from 0.01 to 25% by weight of nitrite are reacted in the process.

16. A process according to claim 1, wherein the process is carried out at a temperature of from 0° C. to 80° C.

17. A process according to claim 1, wherein the compound of formula II is isolated from the reaction solution in yields of from 60% to 100% by extraction or crystallization or extraction and crystallization.

18. A process according to claim 1, wherein the yield of the compound of formula II is from about 90% to 100%, based on the amount of the compound of formula I.

19. A process according to claim 1, wherein the overall activity is from 0.1 up to 5 U/mgₙᵢₜᵣᵢₗₐₛₑ.

20. A process according to claim 19, wherein the overall activity is from 0.1 up to 0.2 U/mgₙᵢₜᵣᵢₗₐₛₑ.

21. A process according to claim 19, wherein the overall activity is from 0.5 up to 2 U/mgₙᵢₜᵣᵢₗₐₛₑ.

22. A process according to claim 1, wherein the concentration of formula II in the reaction mixture is from 20 g/l to 40 g/l.
